(19) United States
(12) Patent Application Publication
(10) Pub. No.: US 2021/0316154 A1
(43) Pub. Date: Oct. 14, 2021

(54) PROPRIETARY MULTI-WAVELENGTH STEALTH MICRO-PULSED LASER THERAPY TECHNOLOGY

(71) Applicants: Bruce Coren, West Palm Beach, FL (US); Joseph Costello, West Palm Beach, FL (US)

(72) Inventors: Bruce Coren, West Palm Beach, FL (US); Joseph Costello, West Palm Beach, FL (US)

(21) Appl. No.: 16/845,288

(22) Filed: Apr. 10, 2020

(51) Int. Cl. A61N 5/06

(52) U.S. Cl. CPC A61N 5/0613; A61B 2017/00141

(57) ABSTRACT

A method for treating pain using laser therapy is provided. In continuous wave mode, the method comprises a laser receiving a power setting and administering treatments of 810 nm, 910 nm, 1064 nm wavelengths, each of 50 us duration, and each followed by a 5 microsecond pause. When the laser is alternatively configured in pulse mode, the laser repeats, after a computer-generated pause, the first, second, and third laser treatments. In pulse mode, an emission frequency (Hz) determines a beat or rhythm of a pulse mode being used a frequency of pulse is set via provider selection entered via touch screen on the laser with preset times.

### FIELD OF THE INVENTION

The present disclosure is in the laser field of physical medicine and therapy. More particularly, the present disclosure provides systems and methods for the treatment of pain utilizing a high-power class IV medical laser and specific procedures programmed into software executing on the laser that utilizes a range of laser light frequencies.

### BACKGROUND

Lasers used in the physical medicine and therapy field are an effective analgesic, treating pain for numerous ailments. Such treatments are limited in their effectiveness when treating deeply located areas of pain in the body, due at least to the absorption coefficient of wavelengths into the mammalian body mass.

Typically, laser treatments for pain are conducted using one of two energy output modes. One is to run the laser light continuously or in continuous wave (CW) mode. The second is to pulse the laser light in pulse mode (PM), which effectively pauses the laser emissions in given beats and rhythms.

### BRIEF DESCRIPTION OF THE FIGURES

FIG. 1 is a graph of a continuous wave procedure according to an embodiment of the present disclosure.

FIG. 2 is a graph of a pulse mode procedure according to an embodiment of the present disclosure.

### DETAILED DESCRIPTION

Systems and methods described herein provide programed procedures drawing from data evidencing that specific wavelengths have a more desirable clinical effect than others. A selection of specific laser light wavelengths is provided herein at specific beats and rhythms. The wavelengths are emitted into the body's tissues to decrease pain and inflammation while stimulating (photobiomodulation) cellular function. Systems and methods provided herein are predicated on empirical data regarding the body's response to laser light wavelengths.

A laser treatment is provided with specific frequencies in configured sequences that optimizes analgesic effects of the laser treatment method. The treatment may optimize ATP (Adenosine Triphosphate) energy transfer between cells and thereby achieves a desirable clinical effect in the body's tissues effectively relieving pain and inflammation from deep seated pathologies.

For the continuous wave (CW) method, a class IV laser emits in three different wavelengths. The sequence begins with 810 nm (50 us, Ton), followed by 910 nm (50 us, Ton), followed by 1064 nm (50 us, Ton). Pauses of 5 us (microsecond) follow each emission. A power setting in W (Watts) is determined by the physician and is dependent on the specific patient's condition. In an embodiment, a continuous wave equation is as follows:

810 nm (50 us, Ton) (5 us pause) 910 nm (50 us, Ton) (5 us pause) 1064 nm (50 us Ton) (5 us pause), then repeat.

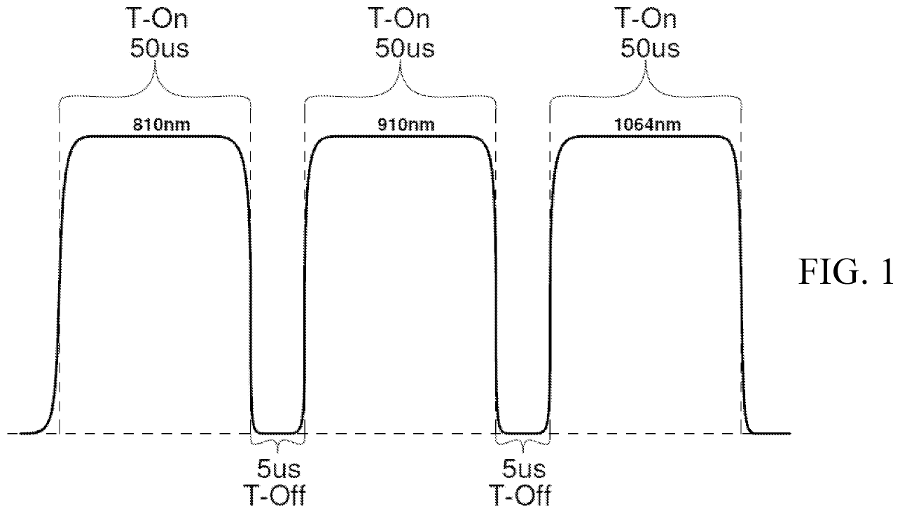

FIG. 1 is a depiction of the continuous wave procedure with the wavelength values shown at the apex of each wave in the vertical column and with the time value represented along the horizontal line at the base of the drawing with a dashed line extending vertically to define the time period.

For the pulse mode method, a class IV laser emits in three different wavelengths. The sequence with 810 um, followed by 910 um, followed by 1064 um. There is a pause between each emission. T-off is automatically computer generated based upon selected HZ and Ton selected by the provider.

As with the continuous wave method the physician will select the suitable power (W) setting for the required treatment, then the physician will determine the length (T-on) of the emission pulse in seconds (s), milliseconds (ms), microseconds (us), after which the physician will then determine an emission frequency (Hz). The (T-off) is calculated by the software and is determined by the (T-on) and the (Hz) measurements. The Hz determines the beat or rhythm of the pulse mode being used. Pulse mode is typically directed to focal pathologies. In an embodiment, a pulse mode equation may be as follows:

810 nm (T-On, Rx) (Toff) 910 nm (T-On, Rx) (Toff) 1064 nm (T-On, Rx) (T-Off calculated by software, quench period), then repeat based upon selected Hz (pulses per second).

In pulse mode, T-On and Hz is based upon several clinical determinations. The laser is configured for a specific patient and their pathology. T-Off is calculated by the software based upon the selected T-On and the number of pulses per second (Hz) determined by the provider.

The duration (T-on) of the frequency pulse is set via a touchscreen on the laser device (Sec, ms, us).

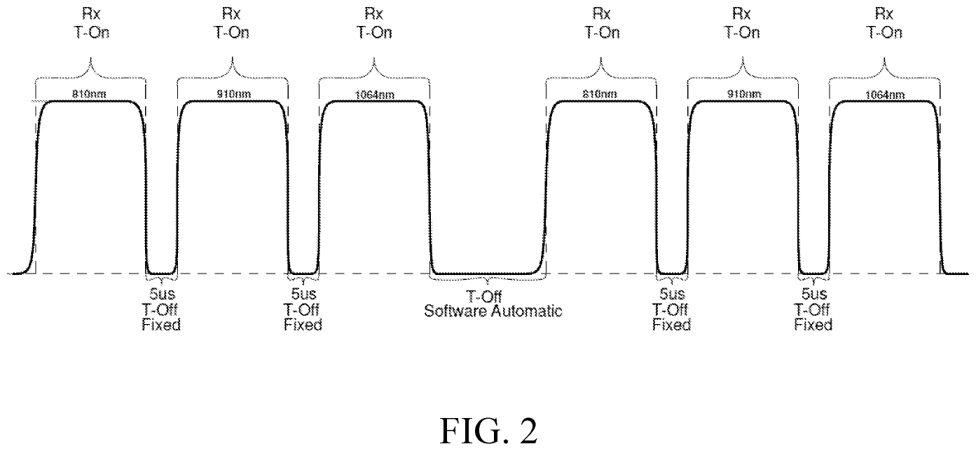

FIG. 2 is a depiction of the Pulse Wave procedure with the wavelength values shown at the apex of each wave in the vertical column. Time value is represented along the horizontal line at the base of the drawing with a dashed line extending vertically to define the time period.

In an embodiment, a 980 nm laser is available that may be used in treating various ailments. The 980 nm laser uses the same software used in other applications of systems and methods provided herein. In embodiments, the 980 nm emits laser with only the 980 nm wavelength.

Systems and methods provided herein may have additional applications. The sequence of wavelength emissions interrupted by a 5 us pause as described above may be in a single (or dual) wavelength emission. This single (or dual) wavelength application is used for both continuous waves as well as pulsed wave treatments. A difference from the multiwavelength treatment is in the application of a single (or dual) wavelength as opposed to the three wavelengths described previously.

What is claimed is:

1. A method for treating pain using laser therapy in continuous wave mode comprising:
a laser receiving a power setting;
the laser administering a laser treatment of 810 nm wavelength of 50 us duration followed by a 5 microsecond pause;
the laser administering a laser treatment of 910 nm wavelength of 50 us duration followed by a 5 microsecond pause; and
the laser administering a laser treatment of 1064 nm wavelength of 50 us duration.

2. The method of claim 1, wherein the laser is a class IV medical laser.

3. The method of claim 1, wherein the power setting is configured in watts.

4. The method of claim 1, wherein the power setting is dependent upon a specific treatment being applied.

5. The method of claim 1, wherein wavelength sequence and configured frequencies of the laser treatments are directed to therapeutic penetration of laser light to deep tissues.

6. The method of claim 1, further comprising the laser effecting (stimulation) adenosine triphosphate (ATP) (at a cellular level).

7. A method for treating pain using laser therapy in pulse mode comprising:
a laser receiving a power setting;
the laser administering a first laser treatment of 810 nm wavelength of a first duration (Ton, Rx) followed by a computer generated (Toff).
the laser administering a second laser treatment of 910 nm wavelength of a second duration (Ton, Rx) followed by computer generated (Toff) the laser administering a third laser treatment of 1064nm wavelength of a third duration (Ton, Rx), followed by a computer generated quench period (Toff).

8. The method of claim 7, wherein the first, second, and third durations are based at least upon a specific treatment being applied for a patient's condition.

9. The method of claim 7, wherein an emission frequency (Hz) associated with the laser treatments is configurable.

10. The method of claim 9, wherein the emission frequency (Hz) determines a beat or rhythm of a pulse mode being used.

11. The method of claim 7, wherein the computer configurable pause is calculated by an application executing at least on the laser.

12. The method of claim 9, wherein the configurable pause is based at least partially on the first, second, and third durations (Ton) and the emission frequency (Hz) configurations.

13. The method of claim 7, wherein the laser is a class IV medical laser.

14. The method of claim 7, wherein a frequency of pulse is set via provider selection entered via touch screen on the laser with preset times.

15. The method of claim 7, wherein the pulse mode is directed to focal pathologies.

16. A system for treating pain using laser therapy comprising:
a class IV medical laser configured for continuous wave mode that:
receives a power setting;
administers a first laser treatment of 810 nm wavelength of 50 us duration followed by a 5 microsecond pause;
administers a second laser treatment of 910 nm wavelength of 50 us duration followed by a 5 microsecond pause; and
administers a third laser treatment of 1064 nm wavelength of 50 us duration.

17. The system of claim 16, wherein when the laser is alternatively configured in pulse mode, the laser repeats, after a computer configured pause, the first, second, and third laser treatments.

18. The system of claim 16, wherein a software application drives the medical laser.

19. The system of claim 16, wherein when the laser is alternatively configured in pulse mode, an emission frequency (Hz) determines a beat or rhythm of a pulse mode being used.

20. The system of claim 16, wherein when the laser is alternatively configured in pulse mode, a frequency of pulse (Hz) is set via provider selection entered via touch screen on the laser with provider-selected times.
